(19) United States
(12) Patent Application Publication
(10) Pub. No.: US 2008/0031971 A1
(43) Pub. Date: Feb. 7, 2008

(54) Use Of Xenon For The Prevention Of Programmed Cell Death

(75) Inventors: Christian Peter Petzelt, Berlin (DE); Wolfgang Johannes Kox, Berlin (DE)

Correspondence Address: WILSON SONSINI GOODRICH & ROSATI, 650 PAGE MILL ROAD, PALO ALTO, CA, 94304-1050, US

(73) Assignee: AGA AB, Lidingo (SE)

(21) Appl. No.: 10/576,628

(22) PCT Filed: Oct. 13, 2004
(86) PCT No.: PCT/EP04/11504

§ 371 Date: Apr. 4, 2007

(30) Foreign Application Priority Data
Oct. 21, 2003 (EP) 03 024 201.0

(51) Int. Cl. A61K 33/00; A61P 43/00

(52) U.S. Cl. 424/600

(57) ABSTRACT

Described is the use of xenon for preventing or reducing cellular death, preferably aberrant apoptosis. Preferred embodiments relate to the use of xenon for preventing (a) cellular damage for tissue and organs to be transplanted, (b) apoptotic cell death after eye laser surgery, and (c) for protecting endothelial cells of the intestine in sepsis.

The present invention relates to the use of xenon for preventing or reducing cellular death, preferably aberrant apoptosis. In particular, the present invention relates to the use of xenon for preventing (a) cellular damage for tissue and organs to be transplanted, (b) apoptotic cell death after eye laser surgery, and (c) for protecting endothelial cells of the intestine in sepsis.

Cell death occurs by necrosis or apoptosis. In necrosis, the stimulus of death induces directly the death of the cell (e.g. by ischemia) whereas in apoptosis the stimulus of death initiates a cascade of events leading in the end, after considerable time, to cellular death. Necrosis is always a pathologic process whereas apoptosis is part of normal development and even essential for normal physiologic function of the organism. However, in addition to that, apoptosis occurs in a variety of diseases and is then called aberrant apoptosis. However, so far the specific treatment of diseases being characterized by an aberrant, pathologically induced apoptosis which is based on the prevention/reduction of aberrant apoptosis is not possible.

Therefore, it is the object of the present invention to provide a therapeutic means for the prevention/reduction of aberrant, pathologically induced apoptosis.

According to the invention this is achieved by the subject matter defined in claim 1. Further advantageous embodiments and aspects of the present invention follow from the dependent claims, the description and the drawings.

During the experiments leading to the present invention it was unexpectedly found that xenon protects neurons from apoptotic cell death induced by apoptosis-inducing substances, i.e. under normoxic conditions. Moreover, by the present inventors it was found that such a protective property of xenon is not limited to neurons. If, for example, human HeLa cells are incubated for several hours with an apoptosis-inducing substance most cells will be thereby committed to apoptotic death and they die after a few hours. If xenon is present during such incubation, cellular death is almost completely prevented. Thus, this property of xenon can be used to protect cells from aberrant, pathologically induced apoptosis.

Xenon is presently known as anaesthetic gas (EP-A-0 864 328; EP-A-0 864 329). Furthermore, it has been reported that xenon may provide some cell protecting effects against neurotransmitter excess (WO-A-00/53192). In addition, it has been reported that xenon administration during early reperfusion reduces infarct size after regional ischemia in the rabbit heart (Preckel et al., Anesthesia and analgesia, December. 2000, 91(6), pages 1327-1332).

Thus, the present invention generally relates to the use of xenon or a xenon gas mixture for the preparation of a pharmaceutical composition for treating (a) aberrant or undesired apoptosis or (b) diseases associated with aberrant apoptosis.

The finding that aberrant or undesired apoptosis can be prevented or at least reduced by xenon opens up a new field of application for this noble gas which has been used so far primarily as inhalation agent in the field of anaesthesia. The prevention or reduction of apoptosis, e.g., characterizing the diseases discussed below, can be carried out according to the present invention on the basis of a simple inhalation therapy. The uptake of xenon via the respiratory system and the transport into the brain are already proven by the use of xenon as an anaesthetic agent. It can also be assumed that the use of xenon has no damaging effect on an organism since many corresponding experiences could be made already by its use as anaesthetic agent. Xenon can be applied by various techniques which can be chosen depending on the particular use. For example, an inhaling apparatus can be used in the clinics, which is already used for anaesthesia by inhalation. If a cardio-pulmonary bypass machine or an other artificial breathing apparatus are used, xenon can be added directly in the machine and requires no further apparatus. On an ambulant basis, e.g., in case of an emergency, it is even possible to use simpler inhalators which mix the xenon with the ambient air during the process of inhalation. In this connection, it is also possible to adapt the xenon concentration and the timing of xenon application in a simple manner to the therapeutic requirements. For example, it might be advantageous to use mixtures of xenon with other gases harmless for humans, e.g., oxygen, nitrogen, air etc.

Examples of preferred uses of xenon are described in the following sections. If “xenon” is mentioned this also includes xenon gas mixtures and it is not intended to restrict the invention to pure xenon.

### (A) Transplantation of Organs and Tissue (Preservation of the Intact Organ/Tissue from the Beginning of Removal, During Transport, up to Re-implantation into the Patient)

During tissue transplantation apoptotic processes are induced that cause (to a varying degree) death in a given cellular population. This can reach up to 95% of all cells, e.g. transfer of human embryonic nigral tissues in Parkinson therapy, intracerebral transplants etc. In the following examples a damage rate of 10-30% of the tissue is—up to the present day—unavoidable and causes considerable increase in the failure rate of transplantation.

(i) The ischemia/reperfusion (I/R) injury is a major problem in liver transplantation or hepatic resection with ischemic procedure, in addition to hepatocyte hypoxemic damage. A burst in production of hydrogen peroxide (H₂O₂) occurring during the reperfusion phase may have a detrimental effect on the organ being reperfused. Immediately after reperfusion, hepatocytes and Kupffer cells generate reactive oxygen species (ROS), including H₂O₂. Subsequently, activated neutrophils, which infiltrate the liver tissue, also produce ROS during the latter phase of reperfusion. H₂O₂-treated cells lead to apoptotic and necrotic death.

(ii) Transplantation of embryonic nigral tissue ameliorates functional deficiencies in Parkinson disease. The main practical constraints of neural grafting are the shortage of human donor tissue and the poor survival of dopaminergic neurons grafted into patients, which is estimated at 5-10%. The required amount of human tissue could be considerably reduced if the neuronal survival was augmented. Studies in rats indicate that most implanted embryonic neurons die within 1 week of transplantation and that most of this cell death is apoptotic. Reduction in cell death immediately after donor tissue preparation and increased long-term graft survival would thus improve dramatically the efficacy of neural transplantation.

(iii) Apoptosis of T-cells, monocytes/macrophages and endothelial cell after heart transplantation. (Transplant-associated coronary artery disease; Induction of apoptosis in lung transplantation)

It is therefore advantageous, if the transport of the tissue to be transplanted and the transplantation itself is performed in the presence of xenon. Xenon can be administered either being present in the surrounding atmosphere or in a xenon-saturated buffer solution. The considerable reduction of cellular damage for tissue and organs to be transplanted and the improved transplantation outcome itself are the major progresses based on this invention.

Thus, a preferred use of xenon or a xenon gas mixture is the prevention or reduction of cellular damage of tissue or organs to be transplanted.

### (B) Prevention of Apoptotic Cell Death after Eye Laser Surgery

Photoreactive keratectomy (PRK) and laser-assisted keratectomy is widely used in ophthalmology to correct and adjust defects and deviations of the cornea. Although these techniques are widely used and accepted as safe, almost 30% of the patients experience irregularities after several months caused by programmed cell death of keratocytes. Unavoidable epithelial injury associated to the use of laser-assisted eye surgery induces keratocyte apoptosis. Such keratocyte loss causes cornea malfunction and requires immediate medical intervention. At present, only additional therapeutic surgery can alleviate or cure such corneal pathological situation, repopulation of anterior corneal stroma after de-epithelialization being too slow a process to be considered useful. Such induction of keratocyte apoptosis is also often observed in transplanted human corneas when the intact corneal epithelium is regenerated, the negative consequences result finally in a considerable percentage of failure to successfully accept the transplant.

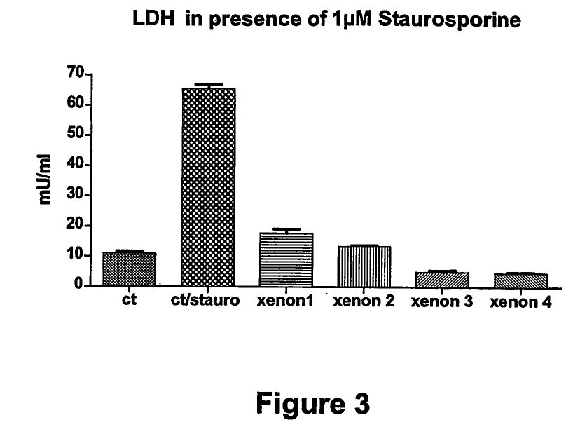

As a preventive measure xenon can be applied immediately after surgery in the form of an air-tight chamber filled with xenon or xenon gas mixtures that is fixed to the eye for preferably some hours (=post-surgery treatment). Alternatively and using the same chamber setup, the eye can be incubated in a xenon atmosphere immediately before surgery for a period of 1-4 hours to reduce the subsequent rate of apoptosis. The beneficial effect of such a pre-treatment is shown in FIG. 3.

Thus, a further preferred use of xenon or a xenon gas mixture is the prevention or reduction of apoptotic cell death after eye laser surgery.

### (C) Protection of Endothelial Cells of the Intestine in Sepsis

Sepsis represents a catastrophic breakdown of the organism often characterized by multi-organ failure, fulminant inflammation and general breakdown of the immune defence mechanisms. Mortality is extremely high and therapeutic possibilities are severely limited. One typical feature is a rapid induction of apoptosis in the endothelial cells of the intestine. As a consequence, this important barrier breaks down resulting in flooding the organism with toxins and immunogens, in a situation where defence mechanisms are already weakened. If in such a situation the induction of apoptosis could be prevented or its rate reduced, valuable time for additional therapies could be gained and mortality could be reduced.

Even in an early stage of sepsis the patient is in a critical situation where it is of importance to gain hours for further treatment. Therefore, an immediate exposure of the endothel of the intestine is to be started in order to prevent the induction of the programmed cell death, respectively the further progress of programmed cell death. The exposure to xenon may be carried out either by introduction of xenon directly into the intestine as gas (due to the high local concentration only a small volume of the gas is to be needed) or by using a xenon-saturated salt solution as described below. In the latter case the application may be achieved either directly parenterally or indirectly via the stomach.

Thus, a further preferred use of xenon or a xenon gas mixture is the protection of endothelial cells of the intestine in sepsis.

In the case of acute life threatening states, e.g., sepsis, respiration can advantageously be carried out with a xenon-oxygen mixture of 90:10% by volume, preferably 80:20% by volume, most preferably 75-70:25-30% by volume, over several hours to one day. As compared thereto, the intermittent respiration by a xenon-air mixture to which less xenon has been added, e.g., 5 to 30% by volume of xenon, preferably 10 to 20% by volume of xenon, can be considered in chronic progression of a disease.

Various methods for the inhalation of xenon and xenon mixtures, respectively, can be used depending on the respective use intended. In clinics, it is possible to use an anaesthetic apparatus, in which pressure vessels containing prefabricated xenon-oxygen mixtures can be connected to the corresponding inlets of the apparatus. The respiration is then carried out according to a procedure common for such apparatus. The same applies analogously to a cardio-pulmonary bypass machine.

As an alternative, xenon can be mixed with ambient air instead of oxygen in the mobile use, which due to the smaller size of the required pressure vessels increases the mobility of the apparatus. For example, it is possible to use an inhalator which supplies xenon from a pressure vessel and is accommodated in a support together with the latter to a mixing chamber. On one side, this mixing chamber contains a mouthpiece for inhaling the xenon and on the other side on which the xenon is supplied to the mixing chamber it has at least one additional check valve which enables the inlet of ambient air. The xenon pressure container can be equipped with a pressure reducing valve, e.g., a valve which reduces the amount of xenon gas supplied to a suitable value. When the patient breathes in, he sucks in air from air valves. In the mixing chamber, this air is mixed with the supplied xenon to the desired ratio and then inhaled by the patient. An advantageous inhalator suitable for mobile use and serving for inhaling xenon and its mixtures is, e.g., described in EP-B1 0 560 928.

For self-medication a mouthpiece can be connected directly to the xenon pressure vessel. During inhalation the patient opens the pressure valve and inhales xenon simultaneously with the air from the environment. When the patient breathes out, she/he releases the valve, so that no more xenon reaches the mouthpiece. In this way, at least a rough regulation of the amount of inhaled xenon is possible.

Alternatively, e.g., for preventing cellular damage of tissues/organs to be transplanted, xenon can be administered as a xenon-saturated solution. A buffered physiologic salt solution (Petzelt et al., Life Sci. 72, 1909-1918 (2003)) is exposed to 100% xenon, or alternatively 80% xenon/20% oxygen, in an air-tight plastic bag and mixed for one hour on a shaker. The gas atmosphere is changed at least one time and the mixing procedure is repeated. Then a complete saturation of the buffer with the gas (mixture) is achieved. This solution is particularly useful for transplatation purposes. If the tissue/organ is maintained during transpaort or during the pre-operation phase in such a solution, a considerable reduction of the rate of apoptosis in the organ/tissue can be observed.

To the above mentioned xenon and xenon gas mixtures also helium may be added. Since helium is a molecule of small size it may function as carrier for the more voluminous xenon. Furthermore, further gases having medical effects may be added, e.g. NO, CO or CO₂. In addition, depending on the disease to be treated other medicaments which are preferably inhalable may be added, e.g. cortisons, antibiotics etc.

### BRIEF DESCRIPTION OF THE DRAWINGS

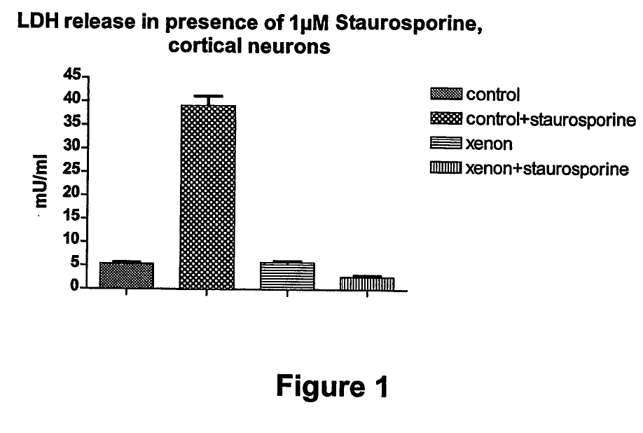

FIG. 1: Induction of apoptosis by staurosporine in cortical neurons
Whereas under normoxic conditions apoptosis occurs, as measured by the release of LDH, xenon prevents completely cell death.

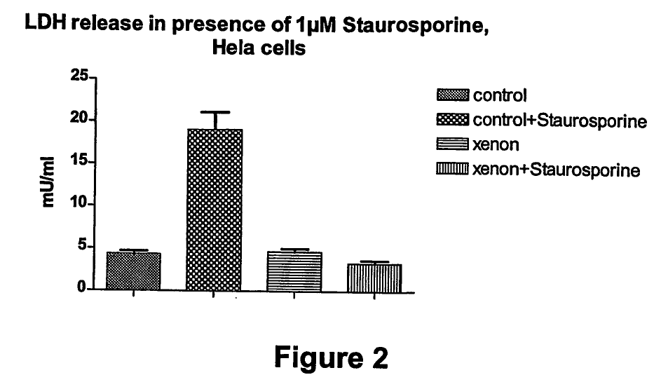

FIG. 2: Induction of apoptosis by staurosporine in HeLa cells
Whereas under normoxic conditions apoptosis occurs, as measured by the release of LDH, xenon prevents completely cell death.

FIG. 3: Effect of pretreating HeLa cells with xenon in order to prevent apoptosis, subsequently induced by staurosporine under normoxic conditions
ct: 4 hrs control medium, 1 hr salt solution;
ct/stauro: 4 hrs control-medium+1 μM staurosporine, 1 hr salt solution+1 μM staurosporine;
xenon 1: 1 hr xe-medium in xenon, 3 hrs control medium+1 μM staurosporine, 1 hr salt solution+1 μM staurosporine;
xenon 2: 2 hrs xe-medium in xenon, 2 hrs control medium+1 μM staurosporine, 1 hr salt solution+1 μM staurosporine;
xenon 3: 3 hrs xe-medium in xenon, 1 hr control medium+1 μM staurosporine, 1 hr salt solution+1 μM staurosporine;
xenon 4: 2 hrs xe-medium in xenon, 2 hrs xe-medium+1 μM Staurosporine, 1 hr salt solution+1 μM staurosporine.

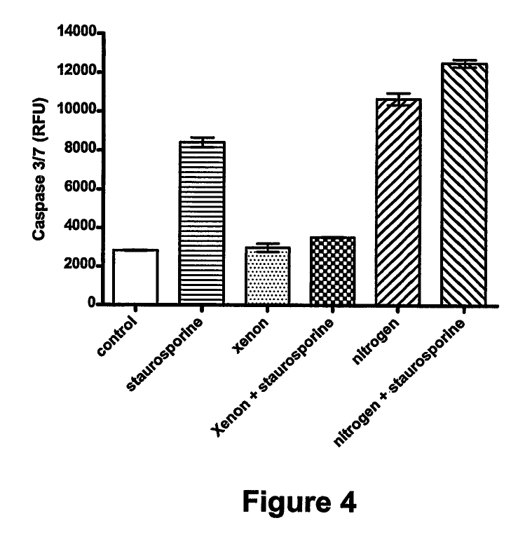

FIG. 4: Activation of caspase 3/7 in HeLa cells after treatment with staurosporin
Control: 5 hrs control medium, 1 hr salt solution
Staurosporine: 5 hours 1 μM Staurosporine.
Xenon: 5 hours Xenon-saturated medium, in Xe
Xenon+Staurosporine: 5 hours Xenon-saturated medium+1 μM staurosporine
Nitrogen: 5 hours N₂-saturated medium in N₂
Nitrogen+Staurosporine: 5 hours N₂-saturated medium in N₂+1 μM staurosporine

The following Examples illustrate the invention.

### EXAMPLE 1

### Methods

### (A) Cells

Rat cortical neurons were obtained from 15-old embryos and maintained for 6 days in vitro as described (Petzelt et al., 2003, Life Sci. 72 (2003), 1909-1918). Human HeLa cells were maintained routinely as monolayer cultures in MEM medium, supplemented with 10% fetal calf serum, 2 mM glutamine, 1% non-essential amino acids. Cultures were subcultivated every two to three days. Absence of mycoplasma was verified every two weeks.

### (B) Induction of Apoptosis

Apoptosis was induced using staurosporine. Staurosporine is an antibiotic originally discovered by Omura et al., J. Antibiot. 30 (1977), 275. It is generally considered a model apoptosis inducer when present in micromolar concentration (Tamaoki et al., BBRC 135 (1986), 397; Nakano et al., J. Antibiot. 40 (1987), 706; Ruegg and Burgess, TIPS 10 (1989), 218; Bertrand et al., Exp. Cell Res. 211 (1994), 314; Wiesner and Dawson, CLAO J. 24 (1996), 1418; Boix et al., Neuropharmacology 36 (1997), 811; Kirsch et al., J. Biol. Chem. 274 (1999), 21155; Chae et al., Pharmacol. Res. 42 (2000), 373; Heerdt et al., Cancer Res. 60 (2000), 6704; Bijur et al., J. Biol. Chem. 275 (2000), 7583; Scarlett et al.,. FEBS Lett. 475 (2000), 267; Tainton et al., BBRC 276 (2000), 231; Tang et al., J. Biol. Chem. 275 (2000), 9303; Hill et al., J. Biol. Chem. 276 (2001), 25643). Cells were seeded in 24-well plates at 6 days before the experiment (for cortical neurons), respectively two days (for HeLa cells) and incubated for several hours in the respective medium containing 1 μM staurosporine, followed by a further 1-hour incubation in a physiologic salt solution (Petzelt et al., 2003). Cellular damage after the experiment was assessed by measuring spectrophotometrically the concentration of LDH in the original supernatant, before the addition of perchloric acid (Roche Diagnostics, Mannheim, Germany). For the determination of the effect of xenon, cells were maintained for the time period indicated in xenon-saturated solution (medium or salt solution) within a gas-tight incubator filled with xenon (Petzelt et al., 2003).

### EXAMPLE 2

### Xenon Completely Prevents Staurosporine Induced Apoptosis in Cortical Neurons

Cortical neurons were incubated for four hours in medium containing 1 μM staurosporine, followed by an additional 1-hour incubation in salt solution, also containing staurosporine. Control preparations were treated in exactly the same way, except that no staurosporine was present. Xenon incubation was performed as described above. As seen in FIG. 1, the control cells survive well under the experimental conditions, no appreciable amount of LDH is released. However, if staurosporine is present, considerable cellular damage is observed as measured by the release of LDH. If cells are maintained in xenon-saturated medium, respectively salt solution, within a xenon-saturated atmosphere, they also survive well the treatment, no difference to cells maintained in a normoxic atmosphere is found. Surprisingly, if the same incubation is performed in the xenon-containing environment but in the presence of 1 μM staurosporine, no cellular damage is found, in contrast to the cells maintained under normoxic conditions. The entrance into apoptosis is prevented.

### EXAMPLE 3

### Xenon Completely Prevents Staurosporine Induced Apoptosis in HeLa Cells

In order to test if the apoptosis-reducing effect of xenon described in Example 2 is restricted to neurons or if it may be considered as a more general phenomenon, human HeLa cells were tested under identical conditions as described in Example 2. HeLa cells are cells derived from a human uterus carcinoma, therefore a sufficient basis for discrimination was given (different species, completely unrelated tissue).

As seen in FIG. 2, basically the same results are obtained as with cortical neurons. Apoptotic cell death is induced by staurosporine under normoxic conditions, whereas it is completely suppressed in the presence of xenon.

In a more discriminating analysis the activation of the terminal effector caspases 3/7 were analysed after treatment with staurosporine. Caspases are universal proteases, their intracelllular cascade of activation form the central component of apoptosis (Slee, E. A. et al. (1999). Cell Death and Fiffer. 6 : 1067-1074). Basically, the signalling “initiator” caspases and the “effector” caspases can be differentiated. Furthermore, individual caspases can be identified by their specificity for a given substrate consisting of a four to five amino acid sequence (Kumar, S. (1999). Cell Death and Differ. 6: 1060-1066; Thornberry, N. A. et al. (1997). J. Biol. Chem. 272: 17907-17911)

In the following experiment the activation of caspase 3/7 is investigated using a highly sensitive and specific fluorogenic inhibitor of a given activated caspase (Ekert, P. G et al (1999), Cell Death and Differ. 6: 1081-1086). The resulting fluorescence signal is a direct measure of the amount of active caspase and can be analyzed by conventional fluorometry.

HeLa cells were treated for 5 hours with 1 μM staurosporine and the resulting caspase 3/7-activation was determined using the in situ caspase detection kit of CHEMICON (cat.no. APT403). The activity is expressed in RFU (relative fluorescence units).

FIG. 4 shows that staurosporine induces a steep increase in activated caspase 3/7 that is almost completely suppressed in the presence of xenon. If untreated cells are incubated for five hours in nitrogen, apoptosis—as expressed by the activation of caspase 3/7—is induced and that activation is increased even further by staurosporine. No effect of the 5-hour-incubation itself is found (see control and xenon).

### EXAMPLE 4

### Pretreatment with Xenon Reduces Apoptosis caused by a Subsequent Exposure to Staurosporine

A further important extension of the findings of Examples 2 and 3 was made when it was investigated if a pretreatment with xenon may reduce cellular damage caused by a subsequent exposure to staurosporine under normoxic conditions. Such a situation would render xenon a truly apoptosis-preventive agent since it could be applied before the apoptotic damage was expected to occur.

As shown in FIG. 3, already a 1-hour exposure to xenon-containing medium within a xenon-atmosphere suffices to protect the cells from a subsequent exposure to staurosporine under normoxic conditions. The longer the pretreatment of the cells with xenon lasts, the better the apoptotosis-preventive effect becomes manifest. If xenon is not present (=ct/stauro), considerable cell damage is found.

1-11. (canceled)

12. A method for treating a human having an:
(a) aberrant or undesired apoptosis or
(b) a disease associated with aberrant apoptosis
comprising administering to said human an effective amount of a pharmaceutical preparation comprising xenon or a xenon gas mixture.

13. The method according to claim 12 comprising, preventing or reducing cellular damage of tissue or organs to be transplanted in a human.

14. The method according to claim 12 comprising preventing or reducing apoptotic cell death after eye laser surgery.

15. The method according to claim 12 comprising protecting endothelial cells of the intestine in sepsis.

16. The method according to any one of claims 12 to 15 wherein the pharmaceutical preparation contains 5 to 90% by volume of xenon.

17. The method according to claims 16 wherein the pharmaceutical preparation contains 5 to 30% by volume of xenon.

18. The method according to any one of claims 12 to 15 wherein the pharmaceutical preparation additionally contains oxygen, nitrogen and/or air.

19. The method according to any of claims 12 to 15 wherein the pharmaceutical preparation additionally contains helium, NO, CO, CO₂, other gaseous compounds and/or inhalable medicaments.

20. The method according to claim 16 wherein the pharmaceutical preparation has a ratio of xenon to oxygen of 80 to 20% by volume.

21. A method of preparing a pharmaceutical preparation by mixing xenon with another gas harmless to humans.

22. The method of claim 21 wherein xenon is mixed with an oxygen-containing gas.

23. A pharmaceutical preparation comprising a xenon gas mixture, wherein the xenon to oxygen ratio is 80 to 20% by volume.

24. The pharmaceutical preparation of claim 23 which additionally contains helium, NO, CO, CO₂, and other gaseous compounds and/or inhalable medicaments.
